(19) United States
(12) Patent Application Publication
(10) Pub. No.: US 2022/0409880 A1
(43) Pub. Date: Dec. 29, 2022

(54) INTRAORAL AND FACIAL BIOREGULATORY BIOELECTRIC DELIVERY DEVICES

(71) Applicant: Blair Bittner Toohey, Corona Del Mar, CA (US)

(72) Inventor: Blair Bittner Toohey, Corona Del Mar, CA (US)

(21) Appl. No.: 17/809,427

(22) Filed: Jun. 28, 2022

Related U.S. Application Data
(60) Provisional application No. 63/216,309, filed on Jun. 29, 2021.

(51) Int. Cl. A61N 1/04; A61N 1/36

(52) U.S. Cl. CPC A61N 1/0476; A61N 1/0484; A61N 1/36034

(57) ABSTRACT

Devices for treating sleep apnea or other conditions by bioelectric signaling include intraoral devices and facial masks. An intraoral device may include a flexible tube shaped to fit over a person's mandible or maxilla and extend along the person's oral mucosa on an interior side and an exterior side of the person's mandibular or maxillary arch or may include a plurality of adjustable, flexible arms extending from a base that may be positioned relative to the person's oral mucosa as desired. Facial masks may be shaped to fit over a person's nose and sinuses. A plurality of electrodes may be provided along the flexible tube, on respective flexible arms, and/or on a surface of the mask. An external or onboard processor may be operable to generate a bioelectric signal at one or more of the plurality of electrodes to heal, restore, or maintain the body's own bioregulatory functions.

### CROSS-REFERENCE TO RELATED APPLICATIONS

Not Applicable

### STATEMENT RE: FEDERALLY SPONSORED RESEARCH/DEVELOPMENT

Not Applicable

### BACKGROUND

Continuous positive airway pressure (CPAP) can be an effective therapy for people suffering from sleep apnea or other airway patency disorders. However, patient compliance with CPAP therapy is low due to the obtrusiveness and inconvenience of CPAP machines. Alternative treatments exist, such as mouthguards that position the tongue and/or jaw to prevent the occurrence of sleep apnea. However, some people find such oral appliances just as uncomfortable or more uncomfortable than CPAP machines. Moreover, both CPAP machines and mouthguards require the patient to adopt a permanent lifestyle change. With no end to treatment in sight, many patients opt to forgo or discontinue treatment entirely, choosing instead to continue suffering from sleep apnea or other airway patency disorders to the detriment of their health.

### BRIEF SUMMARY

The present disclosure contemplates various devices and methods for overcoming the above drawbacks accompanying the related art and, more generally, for providing a delivery system for bioelectric signals. One aspect of the embodiments of the disclosure is an intraoral device. The intraoral device may comprise a flexible tube shaped to fit over a person's mandible and extend along the person's oral mucosa on an interior side and an exterior side of the person's mandibular arch, a plurality of electrodes disposed within and spaced apart along the flexible tube, and a processor disposed within the flexible tube and electrically connected to the plurality of electrodes. The processor may be operable to generate a bioelectric signal at one or more of the plurality of electrodes.

The flexible tube may comprise a polymer. The flexible tube may define an interior arch shaped to extend along the person's oral mucosa on the interior side of the person's mandibular arch and an exterior arch shaped to extend along the person's oral mucosa on the exterior side of the person's mandibular arch. The intraoral device may comprise one or more biasing members that bias the interior arch and the exterior arch of the flexible tube toward each other.

The intraoral device may comprise a memory disposed within the flexible tube. The processor may generate the bioelectric signal according to a mode of operation stored in the memory. The intraoral device may comprise a communication interface disposed within the flexible tube. The processor may generate the bioelectric signal according to a command received by the communication interface. The intraoral device may comprise a power switch disposed within or on the flexible tube. The processor may generate the bioelectric signal in response to a switching of the power switch from an OFF state to an ON state.

Another aspect of the embodiments of the present disclosure is an intraoral device. The intraoral device may comprise a flexible tube shaped to fit over a person's maxilla and extend along the person's oral mucosa on an interior side and an exterior side of the person's maxillary arch, a plurality of electrodes disposed within and spaced apart along the flexible tube, and a processor disposed within the flexible tube and electrically connected to the plurality of electrodes. The processor may be operable to generate a bioelectric signal at one or more of the plurality of electrodes.

The flexible tube may comprise a polymer. The flexible tube may define an interior arch shaped to extend along the person's oral mucosa on the interior side of the person's maxillary arch and an exterior arch shaped to extend along the person's oral mucosa on the exterior side of the person's maxillary arch. The intraoral device may comprise one or more biasing members that bias the interior arch and the exterior arch of the flexible tube toward each other.

The intraoral device may comprise a memory disposed within the flexible tube. The processor may generate the bioelectric signal according to a mode of operation stored in the memory. The intraoral device may comprise a communication interface disposed within the flexible tube. The processor may generate the bioelectric signal according to a command received by the communication interface. The intraoral device may comprise a power switch disposed within or on the flexible tube. The processor may generate the bioelectric signal in response to a switching of the power switch from an OFF state to an ON state.

Another aspect of the embodiments of the present disclosure is a bioregulatory bioelectric signaling device. The bioregulatory bioelectric signaling device may comprise a mask shaped to fit over a person's nose and sinuses, a plurality of electrodes disposed within the mask and spaced apart along a surface of the mask, and a processor disposed within the mask and electrically connected to the plurality of electrodes. The processor may be operable to generate a bioelectric signal at one or more of the plurality of electrodes.

The plurality of electrodes may be disposed along the surface of the mask at positions corresponding to the person's maxillary ethmoid and frontal sinuses.

Another aspect of the embodiments of the present disclosure is a method of treating sleep apnea. The method may comprise positioning a flexible tube over a person's mandible or maxilla to extend along the person's oral mucosa on an interior side and an exterior side of the person's mandibular or maxillary arch and generating a bioelectric signal at one or more of a plurality of electrodes disposed within and spaced apart along the flexible tube.

The method may comprise storing a mode of operation in a memory disposed within the flexible tube. The generating may include generating the bioelectric signal according to the mode of operation stored in the memory. The method may comprise receiving a command from an external device. The generating may include generating the bioelectric signal according to the received command.

Another aspect of the embodiments of the present disclosure is a method of treating sleep apnea. The method may comprise positioning a mask over a person's nose and sinuses and generating a bioelectric signal at one or more of a plurality of electrodes disposed within and spaced apart along a surface of the mask.

The method may comprise storing a mode of operation in a memory disposed within the mask. The generating may include generating the bioelectric signal according to the mode of operation stored in the memory. The method may comprise receiving a command from an external device. The generating may include generating the bioelectric signal according to the received command.

Another aspect of the embodiments of the present disclosure is an intraoral device. The intraoral device may comprise a plurality of electrodes for delivering bioelectric signals to a person's oral mucosa and a plurality of flexible arms on which the plurality of electrodes are respectively provided. The plurality of flexible arms may be adjustable to position the respective electrodes in contact with the person's oral mucosa. The intraoral device may further comprise a base from which the plurality of flexible arms extend. The base may route a plurality of wires to the electrodes via the flexible arms. For example, the base may house a flexible printed circuit board (PCB) and the wires may extend therefrom into the respective flexible arms.

The intraoral device may comprise a processor disposed within the base and electrically connected to the plurality of electrodes via the wires. The processor may be operable to generate a bioelectric signal at one or more of the plurality of electrodes.

### BRIEF DESCRIPTION OF THE DRAWINGS

These and other features and advantages of the various embodiments disclosed herein will be better understood with respect to the following description and drawings, in which like numbers refer to like parts throughout, and in which:

FIG. 1 shows an intraoral device according to an embodiment of the present disclosure, together with a person's mandible;

FIG. 2 shows the intraoral device fitted over the person's mandible;

FIG. 3 shows a top view thereof;

FIG. 4 shows a cross-sectional view taken along the line 4-4 in FIG. 2;

FIG. 5 shows a facial device according to an embodiment of the present disclosure, together with a person's face;

FIG. 6 shows the facial device fitted over the person's face;

FIG. 7 shows a side view thereof;

FIG. 8 shows an intraoral device according to another embodiment of the present disclosure, together with a person's maxilla;

FIG. 9 shows the intraoral device with an outer portion removed to show internal components;

FIG. 10 shows a top view of the intraoral device;

FIG. 11 shows a front view of the intraoral device; and

FIG. 12 shows a cross-sectional view taken along the line 12-12 in FIG. 8.

FIG. 13 shows a cross-sectional view taken along the line 13-13 in FIG. 12;

FIG. 14 shows a cross-sectional view taken along the line 14-14 in FIG. 13;

FIG. 15 shows a facial device according to another embodiment of the present disclosure, together with a person's face;

FIG. 16 shows rear view of the facial device; and

FIG. 17 shows a side view thereof, together with the person's face.

### DETAILED DESCRIPTION

The present disclosure encompasses various embodiments of intraoral devices, facial masks, and methods of delivering bioelectric signals using such devices and masks to induce bioelectric signals to induce cellular transcriptions and bioregulatory changes in the body for various purposes including the treatment of sleep apnea and other airway patency disorders. The detailed description set forth below in connection with the appended drawings is intended as a description of several currently contemplated embodiments and is not intended to represent the only form in which the disclosed innovations may be developed or utilized. The description sets forth the functions and features in connection with the illustrated embodiments. It is to be understood, however, that the same or equivalent functions may be accomplished by different embodiments that are also intended to be encompassed within the scope of the present disclosure. It is further understood that the use of relational terms such as first and second and the like are used solely to distinguish one from another entity without necessarily requiring or implying any actual such relationship or order between such entities.

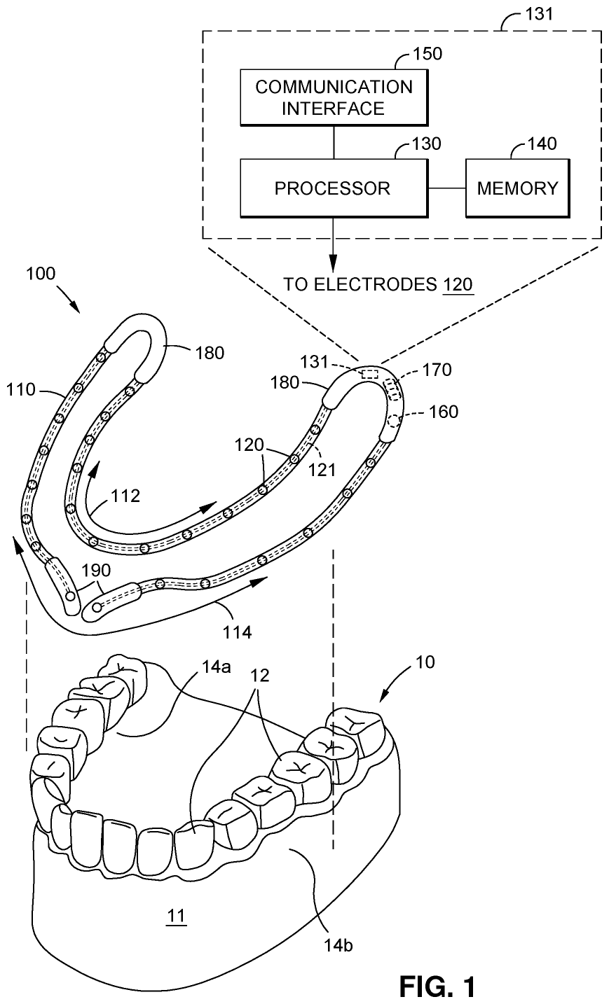

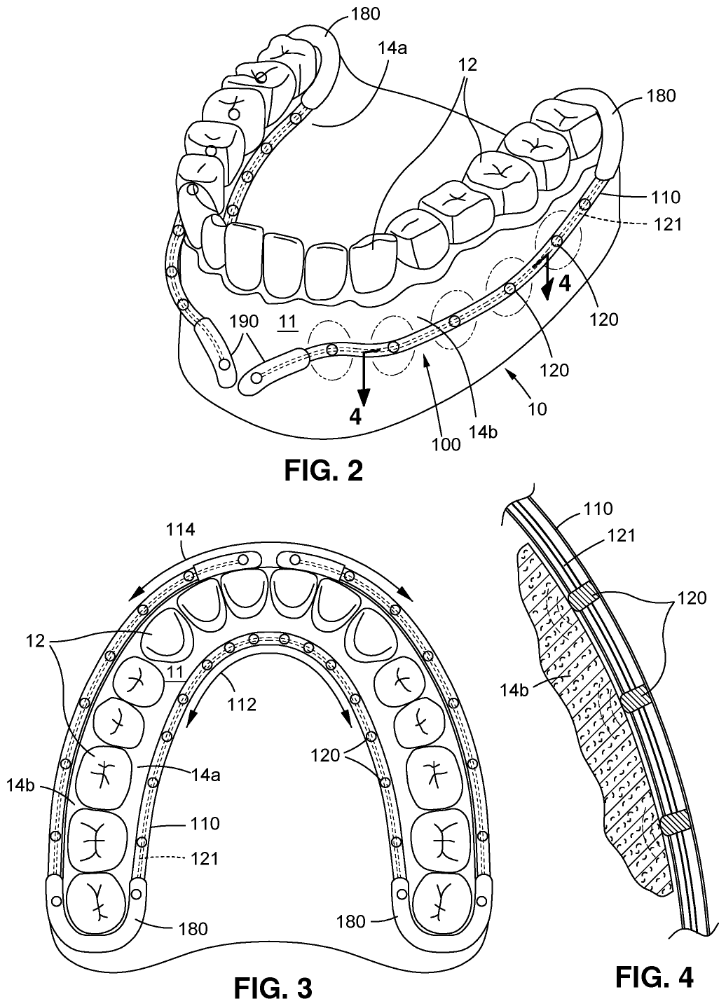

FIG. 1 shows an intraoral device 100 according to an embodiment of the present disclosure together with a person's mandible 10. FIGS. 2-4 show the intraoral device 100 fitted over the person's mandible 10, with FIG. 3 showing a top view thereof and FIG. 4 showing a cross-sectional view taken along the line 4-4 in FIG. 2. The intraoral device 100 may comprise a flexible tube 110 that is placed in a person's mouth and fitted over the person's mandible 10 in contact with the oral mucosa 14a on the interior side of the person's mandibular arch 11 and the oral mucosa 14b on the exterior side of the person's mandibular arch 11 within the vestibule. By generating a current or voltage pattern or other bioelectric signal at one or more of a plurality of electrodes 120 disposed within the flexible tube 110, the intraoral device 100 may apply the bioelectric signal to the person's oral mucosa 14a, 14b as a treatment for airway patency disorders such as sleep apnea or other conditions.

It is believed that the efficacy of bioelectric signaling as a sleep apnea treatment stems from its effects on a person's bioregulatory systems and functions, which may include tissue rejuvenation (e.g., of atrophied muscles), reduction of inflammation, opening of the sinuses and throat, establishing and maintaining airway patency (e.g., in oral and nasal passages), lymphatic drainage, increasing nerve and blood supply, and signaling cellular transcription, as well as pain reduction. Specific examples of treatments and bioelectric signals that may be delivered by the intraoral device 100 for various purposes (not limited to sleep apnea) can be found in U.S. Pat. No. 10,646,644, entitled “Stimulator, Pump and Composition,” U.S. Pat. No. 10,960,206, entitled “Bioelectric Stimulator,” U.S. Pat. No. 7,483,749, entitled “Method of Enhancing Myogenesis by Electrical Stimulation,” U.S. Pat. No. 7,341,062, entitled “Method of Providing a Dynamic Cellular Cardiac Support,” U.S. Pat. No. 10,695,563, entitled “Orthodontic Treatment,” U.S. Patent Application Pub. No. 2020/0000709, entitled “Combination of Bioelectrical Stimulator and Platelet-Rich Fibrin for Accelerated Healing and Regeneration,” U.S. Patent Application Pub. No. 2020/0289826, entitled “Klotho Modulation,” and U.S. Patent Application Pub. No. 2020/0324106, entitled “Bioelectric Stimulation for Sonic Hedgehog Expression,” the entire contents of each of which is expressly incorporated by reference herein. For purposes of treating sleep apnea, it is contemplated that the intraoral device 100 may be worn, for example, for thirty minutes two to three times per week under the supervision of a physician, with the occurrence of sleep apnea in the patient expected to be reduced or eliminated after two to three months as airway patency is reestablished and/or maintained by the bioelectric signals. Unlike conventional treatment options that entail permanent lifestyle changes on the part of the patient, use of the disclosed intraoral device 100 may have the beneficial effect of correcting the patient's bioregulatory systems to allow the body to take control and eliminate the disease progression of sleep apnea. The disclosed intraoral device 100 may thus represent a cure for sleep apnea and other airway patency disorders.

The flexible tube 110 may be made of a polymer such as a conductive polymer, for example, and may be shaped to fit over a person's mandible 10 and extend along the person's oral mucosa 14a, 14b on an interior side and an exterior side of the person's mandibular arch 11. In the illustrated example, reference number 14a refers to the inner oral mucosa (i.e., the oral mucosa on the interior side of the mandibular arch 11), while reference number 14b refers to the outer oral mucosa (i.e., the oral mucosa on the exterior side of the mandibular arch 11 within the vestibule). Advantageously, the flexible tube 110 may be worn on the mandibular arch 11 below the mandibular teeth 12 so as not to contact the teeth 12 themselves, which might otherwise result in damage to crowns, fillings, etc. caused by the applied current or voltage or other bioelectric signal. At the same time, the flexible tube 110 may be entirely 100% contained within the mouth of the patient, without needing to be held by a physician or affixed to an external device and without requiring the patient's mouth to be open or requiring any part of the device 100 to protrude past the patient's lips (though it is also contemplated that an external signal generator may be connected to the device 100 by a wired connection). In this way, the device 100 may be completely oral without being tooth-borne. While not separately illustrated, it should be noted that the flexible tube 110 may instead or additionally be shaped to fit over a person's maxilla and extend along the oral mucosa on interior and exterior sides of the maxillary arch rather than the mandibular arch. In this case, reference number 10 may be understood to depict the maxilla, reference number 11 may be understood to refer to the maxillary arch, and reference number 12 may be understood to refer to the maxillary teeth, with the view simply inverted.

Disposed within the flexible tube 110, the intraoral device 100 may further include a plurality of electrodes 120. The electrodes 120 may be spaced apart along the flexible tube 110 as illustrated, numbering anywhere from four to thirty-two electrodes 120, for example (though more or fewer is also contemplated). As schematically depicted in FIGS. 3 and 4, each electrode 120 may apply the bioelectric signal(s) to a local region of the oral mucosa 14a, 14b where the electrode 120 contacts or comes close to the oral mucosa 14a, 14b. If a conductive polymer is used for the flexible tube 110, the electrodes 120 may be disposed beneath the surface of the flexible tube 110, with only the adjacent outer surface of the flexible tube 110 contacting the oral mucosa 14a, 14b.

The bioelectric signal (e.g., pattern of current/voltage) may be generated by a processor 130 that is disposed within the flexible tube 110 and connected to the plurality of electrodes 120. As shown in FIG. 1, for example, the processor 130 may be included in an integrated circuit 131 such as a microcontroller that is built into the intraoral device 100 and connected to the plurality of electrodes 120 by one or more wires 121. The processor 130 may be operable to generate a bioelectric signal at one or more of the plurality of electrodes 120, for example, by producing a digital output of the integrated circuit 131. In some cases, the digital output of the integrated circuit 131 may further be converted to an analog signal by a digital-to-analog converter (DAC) in order to produce a sinusoidal voltage at the electrodes 120, for example. In implementations where the device 100 is connectable by a wired connection to an external device that handles signal generation, it is contemplated that the processor 130 and/or integrated circuit 131 may in some cases be completely omitted.

The particular bioelectric signal may be selected for its efficacy in treating sleep apnea or another condition or, more generally, for inducing any desired bioelectric changes in the body, and may be predefined for the intraoral device 100. Example bioelectric signals may include biphasic, microcurrent, and Russian current modes configured to stimulate upregulation of various proteins including, by way of example, vascular endothelial growth factor (VEGF), Klotho, stromal cell-derived factor-1 (e.g., SDF-1α or SDF-1A), platelet derived growth factor (PDGF), follistatin, insulin-like growth factor (IGF), osteoprotegerin (OPG), for example. In this regard, the integrated circuit 131 may further include a memory 140 for storing one or more modes of operation corresponding to one or more bioelectric signals to be generated, and the processor 130 may generate bioelectric signals according to the stored modes. The mode(s) of operation stored in the memory 140 may be preprogrammed for the intraoral device 100 and/or configurable by a user such as a physician, who may wish to modify an existing mode of operation or create one from scratch, for example. A physician may, for example, select or configure a mode of operation taking into account the particular patient's anatomy (e.g., jaw size) and condition (e.g., severity of sleep apnea). Selection from among multiple preprogrammed modes of operation and/or configuration of a mode of operation may be achieved by wired or wireless connection to an external device (e.g., using a mobile application), in which case the integrated circuit 131 may further include a communication interface 150 such as a data port or a wireless receiver operable to receive commands from an external device (e.g., via a cable or Bluetooth connection). In this way, the processor 130 may generate bioelectric signals according to a command received by a communication interface disposed within the flexible tube 110 (either wirelessly or by a wired connection). It is also contemplated that such commands for selecting or configuring a mode of operation for generating a desired bioelectric signal may be received by manual user input to the intraoral device 100 itself, such as the pressing of one or more buttons disposed on the device 100. In the simplest case, the intraoral device 100 may include a power switch 160 disposed within or on the flexible tube 110, and the processor 130 may generate a bioelectric signal (such as a single preprogrammed current mode or pattern of voltages) in response to a switching of the power switch 160 from an OFF state to an ON state. In the case of only a simple power switch 160 and a single preprogrammed mode of operation (e.g., for a specific treatment purpose), the communication interface 150 may be omitted, for example. Configuration of such a device 100 may be achieved by replacing the integrated circuit 131, for example.

The bioelectric signal generated by the processor 130 may include a combination of one or more current modes, voltage levels, polarities, designated subsets of electrodes 120 for applying the signal, and/or specified timings (e.g., durations, pulse frequencies) associated with each signal and subset of electrodes 120. More generally, the intraoral device 100 may be used as a delivery system for any desired bioelectric signals, as a non-pharmaceutical, non-surgical treatment for a variety of conditions and/or to heal, restore, or maintain the body's own bioregulatory functions.

The intraoral device 100 may be powered by an onboard power source 170, which may comprise a rechargeable battery (e.g., a lithium-ion battery) and a conductive or inductive charging interface, for example. In this way, power for generating bioelectric signals by the processor 130 and/or receiving commands by the communication interface 150 may be provided within the intraoral device 100, such that the intraoral device 100 may be a small, self-contained device that can be easily manipulated by a physician and worn by a patient without tethering the patient to a larger apparatus, which may allow a degree of mobility to the patient during treatment (and in some cases may allow the patient to freely ambulate while wearing the device 100). In other implementations, the intraoral device 100 may be a passive delivery device for externally generated signals, in some cases requiring no power to function.

As noted above, the flexible tube 110 of the intraoral device 100 may be shaped to fit over a person's mandible 10 (or maxilla) and extend along the person's oral mucosa 14a, 14b on interior and exterior sides of the person's mandibular arch 11 (or maxillary arch). To this end, the flexible tube 110 may define an interior arch 112 (see FIGS. 1 and 3) that is shaped to extend along the person's oral mucosa 14a on the interior side of the mandibular arch 11 (or maxillary arch), as well as an exterior arch 114 that is shaped to extend along the person's oral mucosa 14b on the exterior side of the mandibular arch 11 (or maxillary arch). The flexible tube 110 may thus define a curved path that bounds a generally U-shaped region of space that is roughly planar, with the interior and exterior arches 112, 114 respectively defining inner and outer curved portions of the U-shape.

In order to promote a tight enough fit over the person's mandible 10 (or maxilla) to ensure contact of the flexible tube 110 with the person's oral mucosa 14a, 14b on both sides of the mandibular arch 11 (or maxillary arch), the intraoral device 100 may further include one or more biasing members 180 that bias the interior arch 112 and the exterior arch 114 of the flexible tube 110 toward each other. Each of the biasing member(s) 180 may be a sheath that fits over a length of the flexible tube 110 and is made of a more rigid material than the flexible tube 110, such as a more rigid polymer. By including biasing member(s) 180 at designated curved portions of the flexible tube 110 (such as at the tops of the U-shape that demarcate the transition from the interior arch 112 to the exterior arch 114 as shown), the curved portions can rigidly maintain their curvature in spite of the flexibility of the flexible tube 110. In this way, the biasing member(s) 180 may be arranged such that the flexible tube 110 exiting the biasing member(s) 180 on either side thereof is angled toward itself, that is, such that the interior arch 112 and exterior arch 114 are biased toward each other. The thus biased interior and exterior arches 112, 114 may define a space therebetween that is smaller than the distance between the person's inner and outer oral mucosa 14a, 14b, requiring the interior and exterior arches 112, 114 to be flexed apart to fit over the person's mandible 10 (or maxilla). In this way, a tight fit between the flexible tube 110 and the oral mucosa 14a, 14b can be ensured, promoting good electrical contact with the electrodes 120 therewithin.

Flexing apart of the interior and exterior arches 112, 114 to place the intraoral device 100 over the patient's mandible 10 (or maxilla), or to remove it therefrom, may be conveniently achieved by manually separating end pieces 190 formed at the bottom of the U-shape as shown. The end pieces 190 may be made of the same material as the biasing member(s) 180, for example. When the intraoral device 100 is in its relaxed state (as biased by the biasing member(s) 180), the end pieces 190 may be touching one another to complete the exterior arch 114 of the flexible tube 110. When the end pieces 190 are pulled apart, the space between the interior and exterior arches 112, 114 may accordingly become bigger, allowing the intraoral device 100 to be fitted over the patient's mandible 10 (or maxilla) or removed therefrom. Once the end pieces 190 are released, the relaxed state of the flexible tube 110 (acted upon by the biasing member(s) 180) may bring the interior and exterior arches 112, 114 back toward each other, creating the desired tight fit over the patient's oral mucosa 14a, 14b.

As shown in FIG. 1, one or more of the biasing member(s) 180 may also serve to house the integrated circuit 131, power switch 160, and/or power source 170, as well as any other sensitive electronic components that may be included in the intraoral device 100. Since the biasing member(s) 180 may be more rigid than the flexible tube 110 as described above, housing these components in the biasing member(s) 180 may prevent inadvertent damage that might otherwise be caused to them by the flexing of the flexible tube 110.

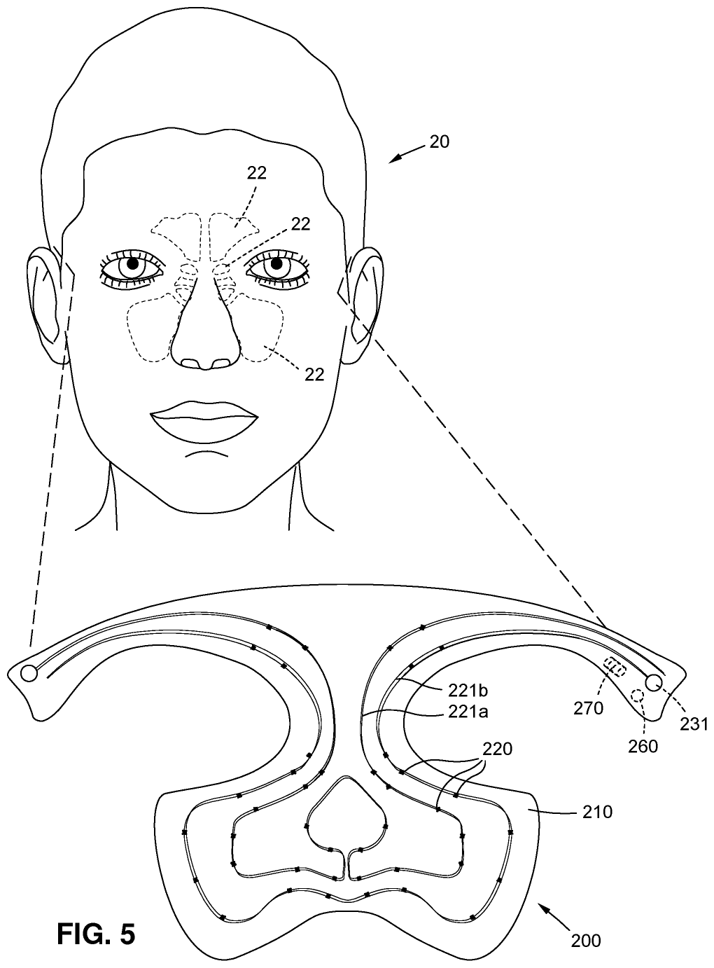

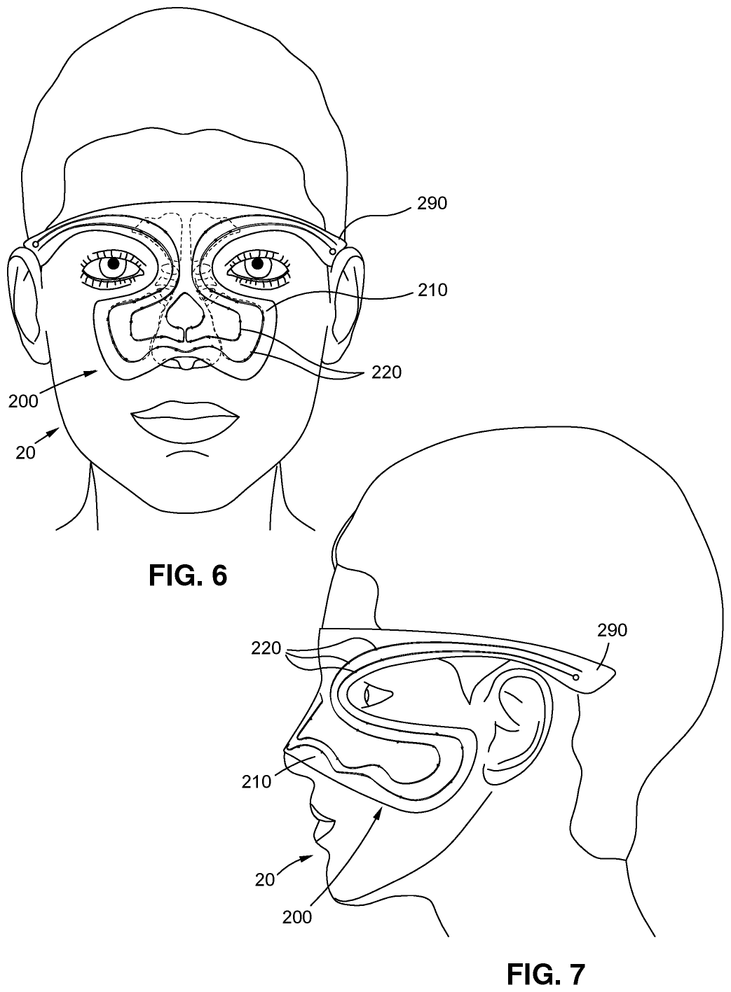

FIG. 5 shows a facial device 200 according to an embodiment of the present disclosure, together with a person's face 20. FIGS. 6 and 7 show the facial device 200 fitted over the person's face 20, with FIG. 6 showing a front view thereof and FIG. 7 showing a side view. Like the intraoral device 100, the facial device 200 may be used to apply bioelectric signaling for the treatment of airway patency disorders such as sleep apnea or other conditions. Whereas the intraoral device 100 may apply bioelectric signals to the person's oral mucosa 14a, 14b as described above, the facial device 200 may apply bioelectric signals to the person's face 20 and, more specifically, the sinuses 22. To this end, the facial device 200 may comprise a mask body or mask 210 that is placed on the person's face 20 and shaped to fit over the person's nose and sinuses 22. In particular, as shown in FIGS. 6 and 7, the mask 210 may be shaped so as to be in contact with a region of the face 20 corresponding to the locations of the sinuses 22, namely extending to the left and right sides of the nose to cover the maxillary sinuses and above the nose to the brow and/or forehead to cover the frontal sinuses. Between these two regions, the mask 210 may extend vertically along the nose between the eyes and thus may cover the ethmoid and sphenoid sinuses as well.

The mask 210 may be a polymer membrane, for example, and may flexibly conform to the shape of the person's face 20. The mask 210 may be held in place by the patient's nose and by friction with the patient's skin, as well as by a pair of temple arms 290 (see FIGS. 6 and 7) that may be adapted to fit over the patient's ears like the temple arms of a pair of glasses. It is also contemplated that the mask 210 may in some cases be treated with an adhesive such as a skin-safe silicone or resin-based adhesive in order to better grip the face 20.

Disposed within the mask 210, the facial device 200 may include a plurality of electrodes 220. The electrodes 220 may be spaced apart along the surface of the mask 210 as illustrated, numbering anywhere from four to thirty-two electrodes 220, for example (though more or fewer is also contemplated). In the illustrated example shown in FIGS. 5-7, the electrodes 220 are provided on two separate lines of wire 221a, 221b, which follow distinct paths through the mask 210 (though they may be part of the same or separate electrical circuits). The use of two or more lines of wire 221a, 221b for the electrodes 220 in this way allows the electrodes 220 to cover a greater area of the surface of the person's face 20. In this regard, it should be noted that the facial device 200 can be thought of as presenting a two-dimensional array of electrodes 220 in order to apply bioelectric signals to the two-dimensional surface corresponding to the locations of the sinuses 22 in the person's face 20, whereas the intraoral device 100 may be regarded essentially as a one-dimensional application since the areas of the oral mucosa 14a, 14b along the mandibular arch 11 (or maxillary arch) are sufficiently narrow that a single row of electrodes 120 is enough. In the case of the facial device 200, each electrode 220 may apply the bioelectric signal(s) to a local region of one of the sinuses 22 where the electrode 220 contacts or comes close to the face 20. As described above in relation to the flexible tube 110, if a conductive polymer is used for the mask 210, the electrodes 220 may be disposed beneath the surface of the mask 210, with only the adjacent outer surface of the mask 210 contacting the face 20.

The precise positions of the electrodes 220 within the mask 210 may be selected to correspond to the maxillary and frontal sinuses, as well as other sinuses such as the sphenoid sinuses. Rather than applying the bioelectric signal(s) to the ethmoid sinuses, it is contemplated that the electrodes 220 may be omitted from the region near the patient's eyes as shown (or that such electrodes 220 may exist but not be used to deliver a signal) in order to avoid arcing and potentially damaging the patient's eyes.

The facial device 200 may further include an integrated circuit 231 that is the same as the integrated circuit 131 of the intraoral device 100 (and may similarly be a microcontroller, for example), along with a power switch 260 and power source 270 that are the same as the power switch 160 and power source 170, respectively. In this regard, the schematic view of the integrated circuit 131 shown in FIG. 1 may equally represent the integrated circuit 231 of FIG. 5, except that the output of the processor 130 is to the electrodes 220 of the facial device 200 rather than to the electrodes 120 of the intraoral device 100 as depicted in FIG. 1. The integrated circuit 231, power switch 260 and power source 270 may be disposed in the mask 210 at any convenient position. As such, the facial device 200 may include a processor 130 disposed within the mask 210 and electrically connected to the plurality of electrodes 220, the processor 130 operable to generate a bioelectric signal at one or more of the plurality of electrodes 220 (e.g., by producing a digital output of the integrated circuit 231). In the illustrated example, the integrated circuit 231, power switch 260, and power source 270 are disposed in one (or both) of the temple arms 290, which may be positioned over the patient's ears. In this way, the physician may easily operate the power switch 260 or any other buttons without obtrusively pressing patient's face 20. In addition, it is contemplated that the temple arms 290 may be required to flex less as they do not need to conform to the unique shape of the patient's face, thus being well suited for housing the sensitive components 231, 260, 270 that might otherwise be damaged by the flexing of the mask 210.

As noted above, two (or more) separate lines of wire 221a, 221b defining the locations of the electrodes 220 may be part of the same or separate electrical circuits. In a case where they are part of the same electrical circuit, there may be one or more electrical connections between the wires 221a, 221b. For example, both wires 221a, 221b may terminate at the same integrated circuit 231, and/or the wires 221a, 221b may be connected together to form a continuous loop. Alternatively, in a case where the wires 221a, 22b are part of separate electrical circuits, there may be a separate integrated circuit 231 (and in some cases a separate power switch 260 and/or power source 270) dedicated to each line of wire 221a, 221b. In this regard, while only a single set of components 231, 260, 270 is illustrated in FIG. 5 (connected to the wire(s) 221b), a matching set of components 231, 260, 270 may be disposed in connection with wire(s) 221a, such as on the opposite side of the mask 210 in the other temple arm 290 (see FIGS. 6 and 7).

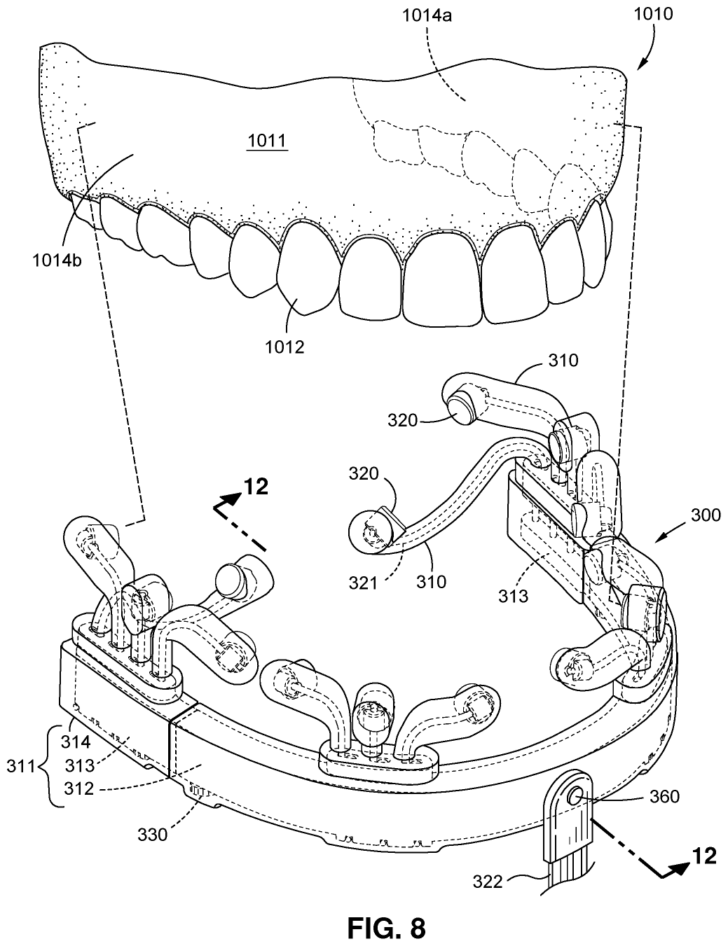

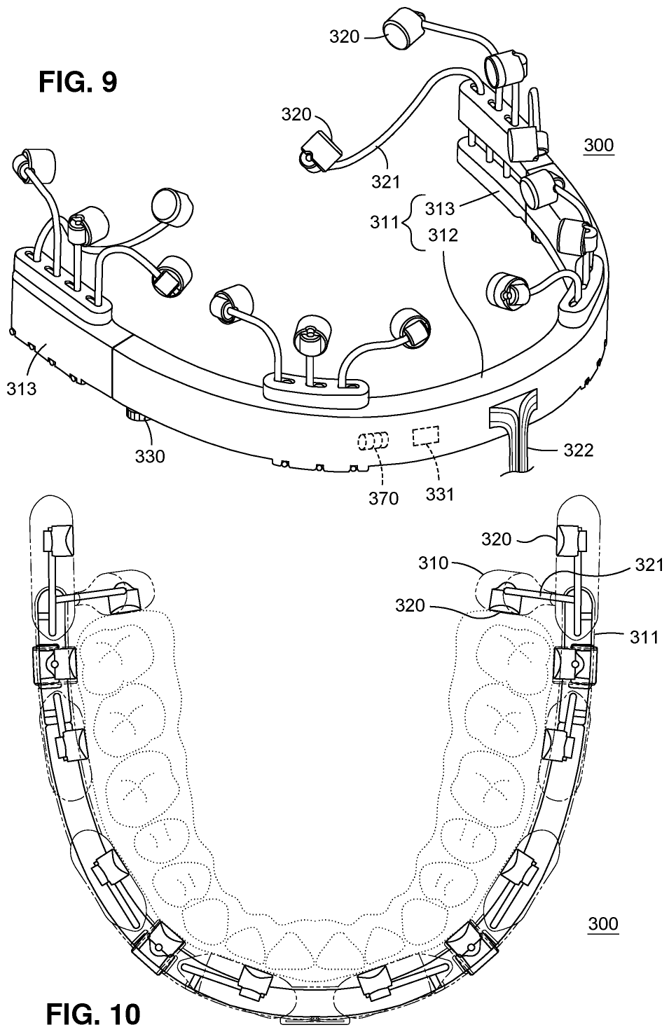

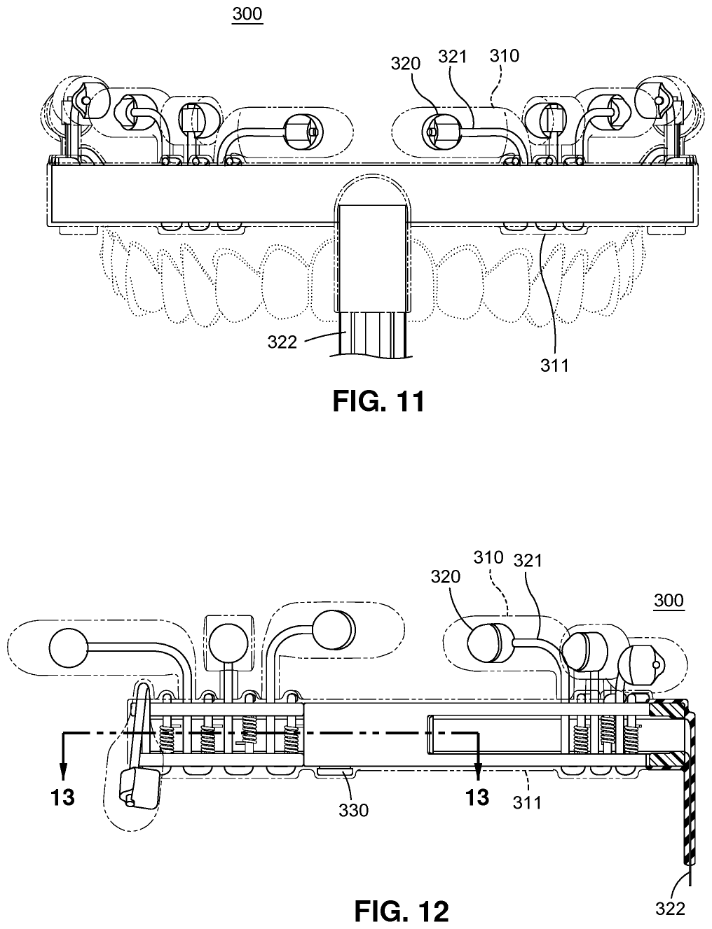

FIGS. 8-12 show an intraoral device 300 according to another embodiment of the present disclosure. FIG. 8 shows the intraoral device 300 together with a person's maxilla 1010. As noted above, reference number 10 of FIG. 1 may also be understood to depict a person's maxilla, with the view simply inverted. In this regard, the maxilla 1010 may be the same as the maxilla represented by the inverted mandible 10 and is only explicitly illustrated here for further clarity in relation to how the intraoral device 300 may be worn. Like the intraoral device 100 described above, the intraoral device 300 may comprise a plurality of electrodes 320 (e.g., stainless steel electrodes) for delivering bioelectric signals to a person's oral mucosa 1014a, 1014b (corresponding to oral mucosa 14a, 14b in FIG. 2). However, whereas the intraoral device 100 may be held in place by the flexible tube 110 that is placed in contact with the oral mucosa 14a, 14b (see FIGS. 1-4), the intraoral device 300 may instead be supported by the electrodes 320 themselves, which may be provided on flexible arms 310 so as to be freely positioned in contact with the oral mucosa 1014a, 1014b according to the person's particular anatomy. The arms 310 may extend from a base 311 that houses a flexible printed circuit board (PCB) 322 connected to wires 321 and/or houses the wires 321 themselves and routes the wires 321 to the electrodes 320 via respective arms 310. The base 311 may conveniently be shaped to fit over the person's maxilla 1010 (or mandible) as shown and may advantageously be held in the mouth by the contact of the electrodes 320 with the oral mucosa 1014a, 1014b, such that the base 311 may “float” near but not in contact with the person's teeth 1012 to avoid damaging them as described above. Thus, like the device 100, the device 300 may be oral without being tooth-borne. As shown in FIG. 8, the base 311 may have a three-piece construction including a front piece 312 and two rear pieces 313 and may further include an outer layer 314 (e.g., silicone), which is removed in FIG. 9.

The arms 310 may be made of a flexible polymer and may embed respective wires 321 for delivering the signals to the electrodes 320. By using a shape-memory alloy such as nickel-titanium for the embedded wires 321, the arms 310 may be made freely adjustable so that the electrodes 320 may be positioned in contact with the person's oral mucosa 1014a, 1014b as desired. The shape-memory alloy may be selected to retain the desired shape with enough stiffness so as to allow the device 300 to be supported by nothing more than the contact of the electrodes 320 with the oral mucosa 1014a, 1014b. That is, the entire device 300 (including the base 311, which should be lightweight), may be able to hold on to the oral mucosa 1014a, 1014b by the electrodes 320 without falling (and without gripping the oral mucosa 1014a, 1014b so tightly as to be painful). As shown in the illustrated example of the device 300, the arms 310 may vary in length, with longer arms 310 being suitable for extension under and around the person's maxilla 1010 (or over and around the person's mandible) in order to place the respective electrodes 320 in contact the oral mucosa 1014a on the interior side of the person's maxillary (or mandibular) arch 1011 in a case where the base 311 is worn outside the teeth 1012. Likewise, in a case where the base 311 is worn on the interior side of the teeth 1012, some of the arms 310 may extend around the person's maxilla 1010 (or mandible) to place electrodes 320 in contact with the oral mucosa 1014b on the exterior side of the person's maxillary (or mandibular) arch 1011. The base 311 of the intraoral device 300 may be generally U-shaped (like the flexible tube 110 of the intraoral device 100) for fitment within the person's mouth while allowing the arms 310 to be positioned close to various points along the oral mucosa 1014a, 1014b.

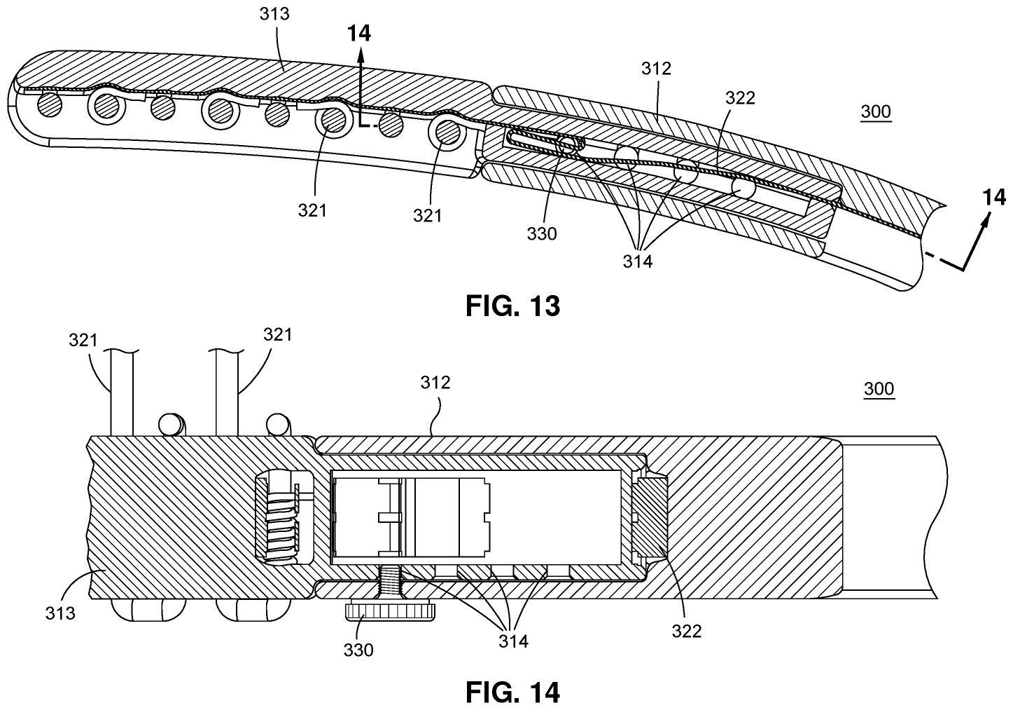

In addition to the adjustable nature of the arms 310, the device 300 may be adjustable to fit a variety of mouth sizes appropriately sizing the base 311. To this end, as noted above, the base 311 may include a front piece 312 and two rear pieces 313, with the front piece 312 being separable from the two rear pieces 313 by a desired distance (e.g., up to one-quarter inch or up to three-eighths inch). To this end, as best shown in FIGS. 13 and 14, each rear piece 313 may slidably fit inside the front piece such that the rear piece 313 can be pushed all the way in (as shown) or pulled out to varying degrees to lengthen the total length of the base 311. While it is contemplated that continuous adjustment may be implemented, the illustrated example shows four discrete adjustment steps corresponding to respective openings 314 formed in the rear piece 313. An adjustment pin 330 may be provided that is arranged to protrude through the front piece 312 and into one of the openings 314 formed in the rear piece 313 to lock the front piece 312 to the rear piece 313 at the position of each discrete adjustment step. When a user wishes to adjust the length of the base 311, the user may simply pinch the base 311 (e.g., pinch the front piece 312 around the position of the adjustment pin 330) at top and bottom (referring to the view in FIG. 14) in order to flex the silicone or other material of the rear piece 313 inward until the adjustment pin 330 comes free of the opening 314. The user can then freely slide the rear piece 313 away from the front piece 312 to lengthen the base 311. By loosening his/her grip on the base 311 (i.e., by releasing the pinch) when the desired length is reached, the user may then allow the rear piece 313 to flex back outward against the adjustment pin 330 such that the adjustment pin 330 enters the nearest opening 314 at the new position. In this regard, it should be noted that the relaxed state of the rear piece 313 may be as shown in FIG. 14, with the rear piece 313 biased toward this relaxed state. As such, the rear piece 313 may have a tendency to spring toward the adjustment pin 330 and allow the adjustment pin 330 to enter the opening 314 as long as the user is not actively flexing the rear piece 313 away from the adjustment pin 330. Note that, as shown in FIG. 13, the flexible PCB 322 (or wires 321) may be given enough slack in order to accommodate the maximum length of the base 311 as it is adjusted.

Like the intraoral device 100, the intraoral device 300 may function as a delivery device for bioelectric signals according to any of various usage scenarios. In particular, the device 300 may be designed to be used with an onboard signal generator and/or with an external signal generator (wired or wireless), depending on how the device 300 is implemented. By way of example, FIG. 8 illustrates that the flexible PCB 322 (or wires 321) may terminate outside the device 300, such as may be the case when the device 300 is designed to be connected to an external signal generator by a wired connection. In the case of an onboard signal generator, the wires 321 and/or flexible PCB 322 may be fully enclosed within the device 300 without protruding and without any wire connector, for example. It is contemplated that the wires 321 and/or flexible PCB 322 may support multiple channel (e.g., 2-channel) capability of the signal generator (whether onboard or external), such that multiple frequencies (e.g., reciprocal or alternating signals) may be delivered simultaneously depending on the desired stimulation.

As shown in FIGS. 8 and 9, the device 300 may further include an integrated circuit 331 that is the same as the integrated circuit 131 of the intraoral device 100 (and may similarly be a microcontroller, for example), along with a power switch 360 and power source 370 that are the same as the power switch 160 and power source 170, respectively. In this regard, the schematic view of the integrated circuit 131 shown in FIG. 1 may equally represent the integrated circuit 331 of FIG. 9, except that the output of the processor 130 is to the electrodes 320 of the intraoral device 300 rather than to the electrodes 120 of the intraoral device 100 as depicted in FIG. 1. The integrated circuit 331, power switch 360 and power source 370 may be disposed in the device 300 at any convenient position. The device 300 may thus include a processor 130 disposed within the device 300 and electrically connected to the plurality of electrodes 320, the processor 130 operable to generate a bioelectric signal at one or more of the plurality of electrodes 320 (e.g., by producing a digital output of the integrated circuit 331). In the illustrated example, the power switch 360 is disposed at the front of the base 311 for easy access by a physician (or the patient).

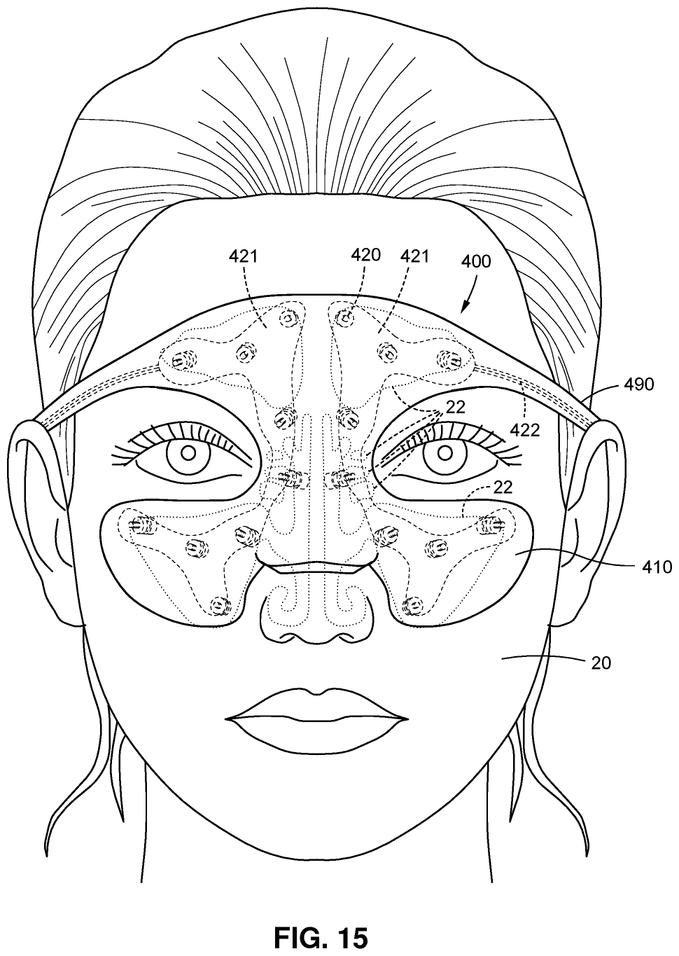

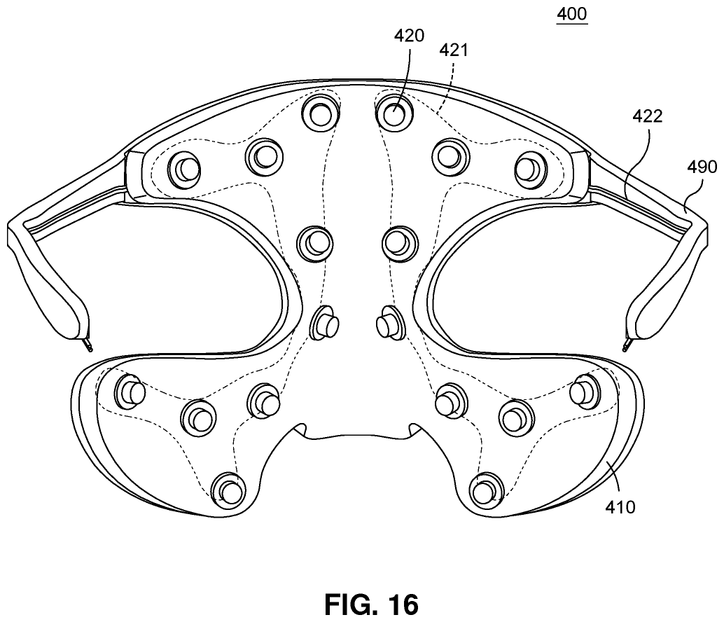

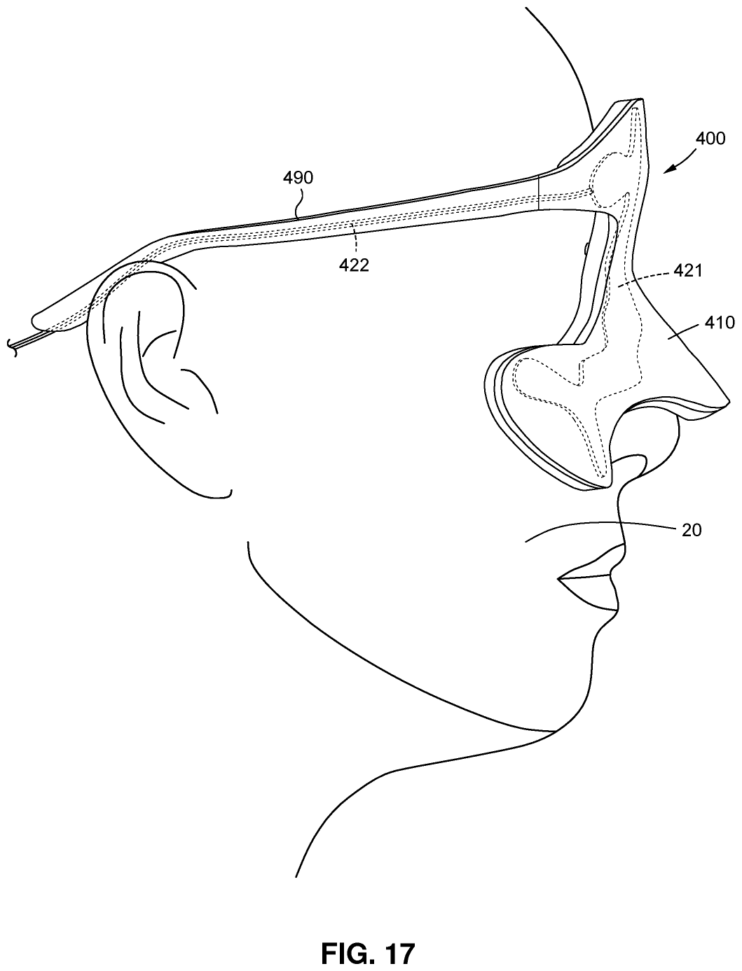

FIG. 15 shows a front view of a facial device 400 according to another embodiment of the present disclosure, together with a person's face 20. FIGS. 16 and 17 show rear and side views thereof, respectively. Like the facial device 200, the facial device 400 may be used to apply bioelectric signals to the person's face 20 and, more specifically, the sinuses 22, for the treatment of airway patency disorders such as sleep apnea or other conditions. To this end, the facial device 400 may comprise a mask body or mask 410, electrodes 420, and temple arms 490, that may be structurally and functionally the same as the mask 210, electrodes 220, and temple arms 290 described in relation to FIGS. 5-7, except as follows. Whereas the illustrated mask 210 of FIGS. 5-7 includes lines of wire 221a, 221b on which the electrodes 220 are provided, the electrodes 420 of the mask 410 shown in FIGS. 15-17 may instead be provided on one or more flexible printed circuit boards (PCB) 421. By using a flexible PCB 421, the electrodes 420 may be freely positioned in two dimensions to cover the desired areas of the person's face 20. In the illustrated example, a pair of flexible PCBs 421 is shown, each connected to input wiring 422 located in the temple arms 490, which may in turn connect to an external and/or onboard signal generator as described herein. In this regard, while not separately illustrated, the facial device 400 may further include the integrated circuit(s) 231, power switch(es) 260, and/or power source(s) 270 of the facial device 200 shown in FIG. 5.

In the above examples of the intraoral device 100, 300 and facial device 200, 400, a plurality of electrodes 120, 220, 320, 420 are used to apply bioelectric signals to a patient's oral mucosa 14a, 14b and/or sinuses 22. Instead of or in addition to the electrodes 120, 220, 320, 420, it is also contemplated that various light therapy modalities may be incorporated into the device 100, 200, 300, 400. In this regard, light-emitting elements such as LEDs, lasers and/or fiber optics may be provided within the device 100, 200, 300, 400 alongside or in place of the electrodes 120, 220, 320, 420 and may similarly be controlled by a processor 130 of an onboard integrated circuit 131, 231, 331. In this way, various benefits of light therapy, including anti-bacterial and anti-viral treatment, increased blood flow, etc. may be realized.

The above description is given by way of example, and not limitation. Given the above disclosure, one skilled in the art could devise variations that are within the scope and spirit of the invention disclosed herein. Further, the various features of the embodiments disclosed herein can be used alone, or in varying combinations with each other and are not intended to be limited to the specific combination described herein. Thus, the scope of the claims is not to be limited by the illustrated embodiments.

What is claimed is:

1. An intraoral device comprising:
a flexible tube shaped to fit over a person's mandible and extend along the person's oral mucosa on an interior side and an exterior side of the person's mandibular arch;
a plurality of electrodes disposed within and spaced apart along the flexible tube; and
a processor disposed within the flexible tube and electrically connected to the plurality of electrodes, the processor operable to generate a bioelectric signal at one or more of the plurality of electrodes.

2. The intraoral device of claim 1, wherein the flexible tube comprises a polymer.

3. The intraoral device of claim 1, wherein the flexible tube defines an interior arch shaped to extend along the person's oral mucosa on the interior side of the person's mandibular arch and an exterior arch shaped to extend along the person's oral mucosa on the exterior side of the person's mandibular arch, the intraoral device further comprising one or more biasing members that bias the interior arch and the exterior arch of the flexible tube toward each other.

4. The intraoral device of claim 1, further comprising a memory disposed within the flexible tube, the processor generating the bioelectric signal according to a mode of operation stored in the memory.

5. The intraoral device of claim 1, further comprising a communication interface disposed within the flexible tube, the processor generating the bioelectric signal according to a command received by the communication interface.

6. The intraoral device of claim 1, further comprising a power switch disposed within or on the flexible tube, the processor generating the bioelectric signal in response to a switching of the power switch from an OFF state to an ON state.

7. An intraoral device comprising:
a flexible tube shaped to fit over a person's maxilla and extend along the person's oral mucosa on an interior side and an exterior side of the person's maxillary arch;
a plurality of electrodes disposed within and spaced apart along the flexible tube; and
a processor disposed within the flexible tube and electrically connected to the plurality of electrodes, the processor operable to generate a bioelectric signal at one or more of the plurality of electrodes.

8. The intraoral device of claim 7, wherein the flexible tube comprises a polymer.

9. The intraoral device of claim 7, wherein the flexible tube defines an interior arch shaped to extend along the person's oral mucosa on the interior side of the person's maxillary arch and an exterior arch shaped to extend along the person's oral mucosa on the exterior side of the person's maxillary arch, the intraoral device further comprising one or more biasing members that bias the interior arch and the exterior arch of the flexible tube toward each other.

10. The intraoral device of claim 7, further comprising a memory disposed within the flexible tube, the processor generating the bioelectric signal according to a mode of operation stored in the memory.

11. The intraoral device of claim 7, further comprising a communication interface disposed within the flexible tube, the processor generating the bioelectric signal according to a command received by the communication interface.

12. The intraoral device of claim 7, further comprising a power switch disposed within or on the flexible tube, the processor generating the bioelectric signal in response to a switching of the power switch from an OFF state to an ON state.

13. A bioregulatory bioelectric signaling device, the bioregulatory bioelectric signaling device comprising:
a mask shaped to fit over a person's nose and sinuses;
a plurality of electrodes disposed within the mask and spaced apart along a surface of the mask; and
a processor disposed within the mask and electrically connected to the plurality of electrodes, the processor operable to generate a bioelectric signal at one or more of the plurality of electrodes.

14. The bioregulatory bioelectric signaling device of claim 13, wherein the plurality of electrodes are disposed along the surface of the mask at positions corresponding to the person's maxillary and frontal sinuses.

15. A method of treating sleep apnea, the method comprising:
positioning a flexible tube over a person's mandible or maxilla to extend along the person's oral mucosa on an interior side and an exterior side of the person's mandibular or maxillary arch; and
generating a bioelectric signal at one or more of a plurality of electrodes disposed within and spaced apart along the flexible tube.

16. The method of claim 15, further comprising:
storing a mode of operation in a memory disposed within the flexible tube,
wherein said generating includes generating the bioelectric signal according to the mode of operation stored in the memory.

17. The method of claim 15, further comprising:
receiving a command from an external device,
wherein said generating includes generating the bioelectric signal according to the received command.

18. A method of treating sleep apnea, the method comprising:
positioning a mask over a person's nose and sinuses; and
generating a bioelectric signal at one or more of a plurality of electrodes disposed within and spaced apart along a surface of the mask.

19. The method of claim 18, further comprising:
storing a mode of operation in a memory disposed within the mask,
wherein said generating includes generating the bioelectric signal according to the mode of operation stored in the memory.

20. The method of claim 18, further comprising:
receiving a command from an external device,
wherein said generating includes generating the bioelectric signal according to the received command.

21. An intraoral device comprising:
a plurality of electrodes for delivering bioelectric signals to a person's oral mucosa;
a plurality of flexible arms on which the plurality of electrodes are respectively provided, the plurality of flexible arms being adjustable to position the respective electrodes in contact with the person's oral mucosa; and
a base from which the plurality of flexible arms extend, the base routing a plurality of wires to the electrodes via the flexible arms.

22. The intraoral device of claim 21, further comprising a processor disposed within the base and electrically connected to the plurality of electrodes via the wires, the processor operable to generate a bioelectric signal at one or more of the plurality of electrodes.
